(19) United States
(12) Plant Patent Application Publication
(10) Pub. No.: US 2016/0242337 P1
(43) Pub. Date: Aug. 18, 2016

(54) Grapevine named "PETIT MERLOT'

(50) ;

(71) Applicants: Università degli Studi di Udine, Udine UD (IT); Istituto di Genomica Applicata, Udine UD (IT)

(72) Inventors: Simone Diego Castellarin, Udine (IT); Guido Cipriani, Faedis (IT); Gabriele Di Gaspero, Cividale del Friuli UD (IT); Michele Morgante, Tricesimo UD (IT); Enrico Peterlunger, Codroipo UD (IT); Raffaele Testolin, Udine (IT)

(73) Assignees: Università degli Studi di Udine, Udine UD (IT); Istituto di Genomica Applicata, Udine UD (IT)

(21) Appl. No.: 14/545,114

(22) Filed: Mar. 27, 2015

(30) Foreign Application Priority Data
Feb. 13, 2015 (QZ) 2015/0365

(51) Int. Cl. A01H 5/00

(52) U.S. Cl. PLT/205

(57) ABSTRACT

A new and distinct variety of grapevine named ‘Petit Merlot’, primarily adapted to the growing conditions of the temperate regions, and characterized by its medium vigor; erect growth habit; medium sized, circular shaped leaves; small, conical, medium dense berry clusters with two middle size wings; blue-black berries with slightly firm flesh, none to herbaceous taste and none to very weak flesh coloration; primary use for wine; and resistance to winter temperatures (to −22° C.), resistance to downy mildew, and tolerance to powdery mildew.

### LATIN NAME OF THE GENUS AND SPECIES OF THE PLANT CLAIMED

Vitis×vinifera (hybrid)

### VARIETY NAME

‘Petit Merlot

### BACKGROUND OF THE INVENTION

The present invention relates to a new and distinct summer/fall bearing grapevine variety, botanically known as Vitis vinifera, and hereinafter referred to by the name ‘Petit Merlot’.

The new grapevine ‘Petit Merlot’ is a product of a controlled breeding program conducted by the inventors in Udine, Italy. The objective of the breeding program was to develop a new grapevine variety particularly characterized by resistance to cold (<−20° C.), resistance to downy mildew (Plasmopara viticola), and tolerance to powdery mildew (Uncinula necator).

The new grapevine ‘Petit Merlot’ originated from a cross made by the inventors in 2002 in Udine, Italy. The female or seed parent is the grapevine variety, Vitis vinifera cv. ‘Merlot’ (unpatented), and the male or pollen parent is the grapevine variety, Vitis cv. ‘20/3’ (Bianca×SK77-4/5) (unpatented).

The new grapevine ‘Petit Merlot’ was discovered and selected by the inventors as a single flowering plant within the progeny of the stated cross in a controlled environment in 2002 in Udine, Italy. Asexual reproduction of the new grapevine variety by grafting was first performed in February 2004 in Rauscedo, Friuli Venezia Giulia region, Italy, and has demonstrated that the combination of characteristics as herein disclosed for the new cultivar are firmly fixed and retained through successive generations of asexual reproduction. The new cultivar reproduces true to type.

### SUMMARY OF THE INVENTION

‘Petit Merlot’ is primarily adapted to the climate and growing conditions of the temperate regions with average yearly temperature about 13° C., minimum winter temperature about −20° C., annual rainfall around 700-1500 mm of rain (e.g. North-Eastern Italy, Friuli) . This region provides the necessary year-round temperatures required for it to produce and maintain a strong vigorous plant with consistent fruit production from April through November on primocanes and in the ensuing year from April through November on the floricanes.

The following traits have been repeatedly observed and are determined to be unique characteristics of ‘Petit Merlot’, which in combination distinguish this grapevine plant as a new and distinct variety.

1. Medium vigor;
2. Erect growth habit;
3. Medium sized, circular shaped leaves with medium to medium green upper surfaces and light green lower surfaces and, medium texture;
4. Small, conical, medium dense berry clusters with two middle size wings; blue-black berries with slightly firm flesh, none to herbaceous taste, and none to very weak flesh coloration; primarily used for wine and maturing in early September (Middle Friuli, North-Eastern Italy);
5. Primocanes and floricanes with a circular cross section, brownish colour, no lenticels, and short-medium internodes (diameter 8 mm);
6. Harvesting time in early September (Middle Friuli, North-Eastern Italy); and
7. Resistance to winter temperature (−22° C.), resistance to downy mildew, and tolerance to powdery mildew.

Plants of the new grapevine ‘Petit Merlot differ from plants of the parents, Vitis vinifera ‘Merlot’ (unpatented) and Vitis cross ‘20/3’ (unpatented), in the following characteristics described in Table 1.

TABLE 1
Comparison with parent varieties

| | | Female Parent | Male Parent |
|---|---|---|---|
| Charac- | New Cultivar | ‘Merlot’ | ‘20/3’ |
| teristic | ‘Petit Merlot’ | (unpatented) | (unpatented) |
| vigor: | medium | medium | medium |
| growth | erect | horizontal | semi-erect |
| habit | | | |
| leaf | small to med- | medium, | Medium size, |
| | ium, medium to | dark green color | light green color |
| | dark green color | (upper surface), | (upper surface), |
| | (upper surface), | green color (low- | green color (low- |
| | light green color | er surface), very | er surface), no |
| | (lower surface), | few hairs in both | hairs in both sur- |
| | no hairs in both | surfaces, wedge | faces, wedge- |
| | surfaces, wedge | shape, medium | shaped-kidney- |
| | shape, medium | texture | shaped, medium |
| | texture, | | texture |
| cluster | low weight, con- | low weight, long, | low weight, conical, |
| | ical with two | with 1-2 wings, | with 1-2 wings, |
| | middle size wings, | medium, berry | medium dense, ber- |
| | loose or medium | skin with blue | ry skin with green- |
| | dense, berry skin | black color, soft | yellow color, slight- |
| | with blueblack | flesh, neutral | ly firm flesh, neu- |
| | colour, soft flesh, | taste, no flesh | tral taste, no flesh |
| | neutral taste, | coloration | coloration |
| | no flesh colora- | | |
| | tion | | |
| primo- | circular cross | circular cross | oblate cross section, |
| cane and | section, brownish | section, brownish | brownish color, |
| floricane | colour, lenticels: | color, lenticels: | lenticels: absent, |
| | absent, short- | absent, short- | medium internodes, |
| | medium inter- | medium inter- | diameter small |
| | nodes, diameter: | nodes, diameter | (about 8 mm) |
| | small (about | small (about | |
| | 8 mm) | 8 mm) | |
| harvest- | First decade | Early-medium | Early (end August, |
| ing time | September (Mid- | early September | Middle Friuli, |
| | dle Friuli, North- | (Middle Friuli, | North-Eastern |
| | Eastern Italy), | North-Eastern | Italy) |
| | | Italy) | |
| resis- | resistant to win- | Average resis- | resistant to winter |
| tances | ter temperature | tance to winter | temperature not |
| | (−22° C.), resis- | temperature (−22° | checked, resistant |
| | tant to downy re- | C.), very low re- | to downy mildew, |
| | sistant to downy | sistance to downy | resistant to pow- |
| | mildew, tolerant | mildew, very low | dery mildew |
| | to powdery mil- | resistance to pow- | |
| | dew. | dery mildew | |

Of the many commercial cultivars known to the present inventor, the most similar to the new grapevine ‘Petit Merlot’ is the female parent ‘Merlot’, to which a comparison has been provided above.

### BRIEF DESCRIPTION OF THE PHOTOGRAPHS

The accompanying photographs illustrate the overall appearance of the new grapevine Petit Merlot showing the colors as true as is reasonably possible with colored reproductions of this type. Colors in the photographs may differ slightly from the color values cited in the detailed botanical description, which accurately describe the color of Petit Merlot.

FIG. 1A and FIG. 1B show a typical fruit cluster of ‘Petit Merlot’ taken on Sep. 9, 2012, in Udine, Italy.

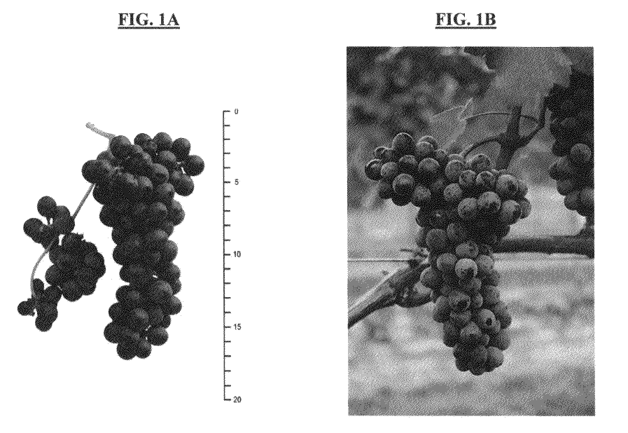

FIG. 2 shows a typical mature leaf (upper surface, left and lower surface, right) of ‘Petit Merlot’ taken on May 31, 2012, in Udine, Italy.

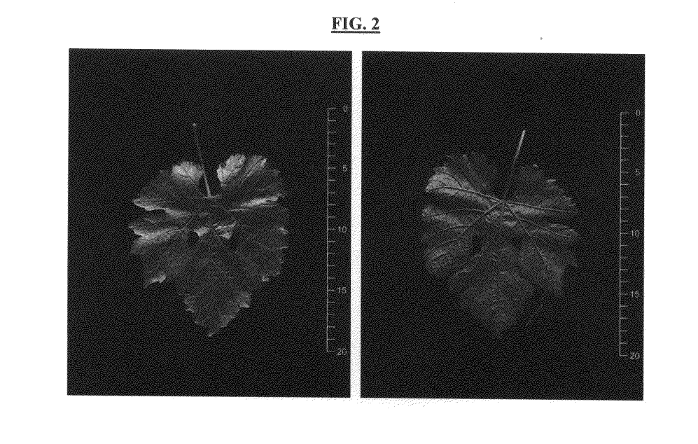

FIG. 3 shows a typical mature vine of ‘Petit Merlot’ taken on Aug. 3, 2012.

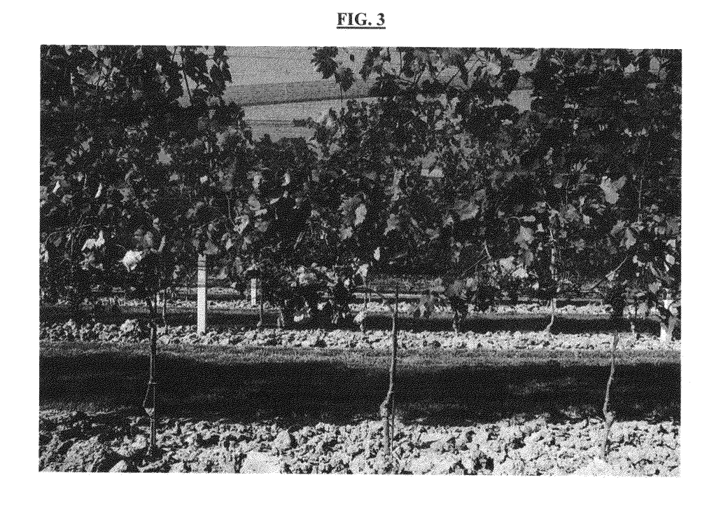

FIG. 4 shows a close-up view of typical mature fruit of ‘Petit Merlot’ taken on Sep. 9, 2012, in Udine, Italy.

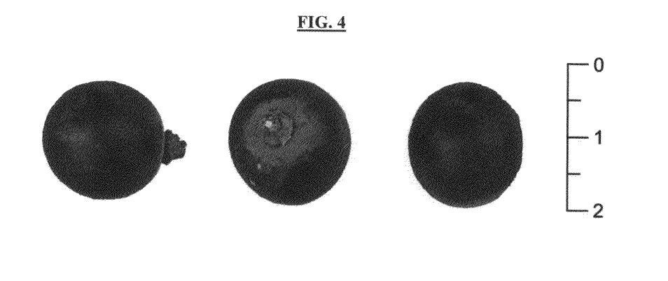

### DETAILED BOTANICAL DESCRIPTION

The following description of ‘Petit Merlot’ unless otherwise noted, is based on observations taken during the 2011, 2012, and 2013 growing seasons(s) in Udine, Italy and Fossalon di Grado (GO), Italy. from plants dug from a nursery located in Rauscedo (PN), Italy during the beginning of December 2007 and planted approximately 16 to 20 weeks later in Udine, Italy and Fossalon di Grado (GO), Italy. The phenotypical descriptions and color designations stated for the new variety may vary, depending upon variations in environmental factors, including weather (temperature, humidity and light intensity), day length, soil type, location and cultural conditions. ‘Petit Merlot’ has not been observed under all possible environmental conditions.

| | | |
|---|---|---|
| | | ‘Merlot’ |
| Characteristic | ‘Petit Merlot’ | (unpatented) |

GENERAL

| | | |
|---|---|---|
| Resistance to | Resistant to downy | Susceptible to downy |
| pest/disease | mildew, tolerant to | mildew and to pow- |
| | powdery mildew | dery mildew |
| Resistance to | Resistant to winter | Resistant to winter |
| natural elements | cold down to −23° C. | cold down to −22° C. |

VINE

| | | |
|---|---|---|
| Vigor | medium | medium |
| Trunk diameter | 6 cm (6 yrs-old plant) | 6 cm (6 yrs-old plant) |
| Bark | | |
| color | Dark brown | Dark brown |
| underbark color | Light brown | Light brown |
| texture | n.a. | n.a. |
| Canes | | |
| length | Internode length | Internode length |
| | cm 10.5 | about cm 10 |
| width | 8 mm | 8 mm |
| Shoots | | |
| shape | Erected, cross section: | Horizontal, cross section: |
| | circular, surface: with | circular, surface: smooth, |
| | stripes, nodes and inter- | nodes and internodes with- |
| | nodes without hairs | out hairs |
| color | internodes with red and | Internodes with green col- |
| | green pigmentation on | our on ventral and dorsal |
| | both sides, nodes with | side, nodes with green ven- |
| | red and green dorsal side | tral and dorsal side |
| | and green ventral side | |
| Tendrils | | |
| form | Bifid or trifid | bifid |
| color | Green | Green |
| texture | normal | normal |
| number | 2 or <2 consecutive | 2 or <2 consecutive |
| Buds | | |
| size | average | average |
| shape | round | round |
| color | Light brown | Light brown |
| number | 2/node | 2/node |
| time of | early | early-medium |
| budbreak | | |

LEAVES

| | | |
|---|---|---|
| Size | medium | medium |
| Number of | n.a. | n.a. |
| leaflets | | |
| Glossiness | Medium | medium |
| Cross section | Twisted | Flat |
| shape | | |
| Color (immature) | | |
| Upper surface | Light green | Light green |
| Under surface | green | green |
| Color (mature) | | |
| Upper surface | Medium green | Dark green |
| Under surface | green | green |
| Petiole | | |
| Length (cm) | 6.2 | 6.0 |
| Color (upper | green | green |
| surface) | | |
| Color (under | green | green |
| surface) | | |
| Stipule | n.a. | n.a. |
| orientation | | |

FLOWERS

| | | |
|---|---|---|
| Flowering period | Early June | Early June |
| (time of beginning | | |
| of flowering) | | |
| Sex | hermaphrodite | hermaphrodite |
| Size | Average | average |
| Diameter (cm) | 0.3 (stamens) | 0.3 (stamens) |
| Fragrance | n.a. | n.a. |
| Flower number | n.a. | n.a. |
| (at 3^{rd} node | | |
| from tip of lateral | | |
| mean and range) | | |
| Petals | | |
| Length (cm) | n.a. | n.a. |
| Width (cm) | n.a. | n.a. |
| Overall shape | calyptra | calyptra |
| Color (immature) | green | green |
| Upper surface | | |
| Under surface | | |
| Color (mature) | green | green |
| Upper surface | | |
| Under surface | | |
| Sepals | | |
| Length (cm) | n.a. | n.a. |
| Width (cm) | n.a. | n.a. |
| Overall shape | n.a. | n.a. |
| Color (immature) | n.a. | n.a. |
| Upper surface | | |
| Under surface | | |
| Color (mature) | n.a. | n.a. |
| Upper surface | | |
| Under surface | | |
| Pedicel | | |
| Length (mm) | 45 (cluster) | 45 |
| Color | green | green |

FRUIT

| | | |
|---|---|---|
| Primocane time | September 6th | September 7th |
| of fruiting (1^{st} | | |
| pick) | | |
| Clusters | | |
| cluster weight | Low (g 160) | Low (g 160) |
| cluster shape | conical | conical |
| cluster length | mm 170 | medium |
| avg. berries | 180 | 180 |
| per cluster | | |
| avg. clusters | 2 | 2 |
| per shoot | | |
| Berries | | |
| Berry size | g 1.67 | g 1.80 |
| Berry length | 1.60 | 1.50 |
| (cm) | | |
| Berry width | 1.60 | 1.50 |
| (cm) | | |
| Overall shape | broad ellipsoid | Broad ellipsoid |
| of berry | | |
| Berry Texture | soft | soft |
| Berry Color | green | green |
| (immature) | | |
| Berry Color | Blue black | Blue black |
| (mature) | | |
| Soluble solids | 22.4 | 22.0 |
| (%) | | |
| Titratable | 5.1 | 5.5 |
| acidity (as g/L | | |
| tartaric acid) | | |
| Sugar/acid ratio | 4.4 | 4.0 |
| Firmness | soft | soft |
| Seeds | 2-3 | 2-3 |
| Skin cracking? | no | rarely |
| Juice color | transparent | transparent |
| Berry taste | Neutral | neutral |
| Eating quality | n.a. | n.a. |
| Berry uses | wine | wine |
| Shipping | n.a. | n.a. |
| quality | | |

What is claimed is:

1. A new and distinct grapevine, referred to as ‘Petit Merlot’, as herein described and illustrated by the characteristics set forth above.
